(19) United States
(12) Patent Application Publication
(10) Pub. No.: US 2002/0055462 A1
(43) Pub. Date: May 9, 2002

(54) Use

(76) Inventors: Michael John Reed, London (GB); Barry Victor Lloyd Potter, Bathford (GB)

Correspondence Address: FROMMER LAWRENCE & HAUG, 745 FIFTH AVENUE- 10TH FL., NEW YORK, NY, 10151, US

(21) Appl. No.: 10/013,798

(22) Filed: Dec. 10, 2001

Related U.S. Application Data
(63) Continuation-in-part of application No. PCT/GB00/02186, filed on Jun. 5, 2000.

(30) Foreign Application Priority Data
Jun. 10, 1999 (GB) 9913536.0

(51) Int. Cl. A61K 31/17; A61K 31/56; A61K 31/337; A61K 31/427

(52) U.S. Cl. 514/2; 514/171; 514/182; 514/365; 514/449

(57) ABSTRACT

There is provided use of a material selected from (i) microtubule stabilizing agent; (ii) microtubule disrupter; (iii) a compound of the formula A-B wherein A is an oxyhydrocarbyl group and B is a cyclic group; and (iv) a compound of the formula C-D wherein C is an sulphamate group and D is a cyclic group, for the manufacture of a medicament for the inhibition of tumor necrosis factor α (TNFα) stimulated aromatase activity.

### RELATED APPLICATIONS

This application is a continuation-in-part application of International Application Number PCT/GB00/02186 filed Jun. 5, 2000, published as WO 00/76487 on Dec. 21, 2000, and claims priority from Great Britain Application Number 9913536.0 filed Jun. 10, 1999.

Reference is also made to the following documents issued to the applicants: U.S. Pat. Nos. 6,239,169, 6,187,766, 6,159,960, 6,083,978, 6,017,904, 6,011,024, 5,861,390, 5,830,886, 5,616,574, and 5,604,215; International Publications WO 0151055, WO 0144268, WO 0076487, WO 0066095, WO 9964013, WO 9927936, WO 9927935, WO 9824802, WO 9732872, WO 9730041, WO 9305064, and WO 9305063; and European Patents EP 0928609, EP 0921130, EP 1099706, EP 1085876, EP 1051178, EP 1051177, EP 0982032, EP 0928609, EP 0885211, EP 0880514, EP 0641355, and EP 0602123.

All of the above-mentioned documents, applications and patents, as well as all documents cited herein and documents referenced or cited in documents cited herein, are hereby incorporated herein by reference.

### FIELD OF THE INVENTION

The present invention relates to inhibition of tumour necrosis factor α stimulated aromatase activity.

### BACKGROUND

Synthesis of oestrone from androstenedione, by the aromatase enzyme complex, is an important source of oestrogen available to support the growth of hormone-dependent tumours (1). Cytokines, such as interleukin 6 (IL-6) and tumour necrosis factor α (TNFα) and Prostaglandin E₂ (PGE₂), can all stimulate aromatase activity (2-4). Many breast tumours are infiltrated by macrophages and lymphocytes and there is evidence that these cells may be an important source of the factors that can stimulate aromatase activity (5-6).

The role that the immune system has in the development of cancers remains controversial (7). In women receiving long-term immune suppressive therapy, however, the incidence of breast cancer is reduced (8). This suggests that the immune system may have an immunostimulatory role in the development of breast cancer. Support for such a role, possibly acting via cytokine stimulation of oestrogen synthesis, was obtained by comparing the abilities of conditioned medium (CM) collected from white blood cells of an immunosuppressed subject or woman with breast cancer to stimulate aromatase (9). Stimulation of the activity of this enzyme was greatly reduced by CM collected from cells of the immunosuppressed subject. Furthermore, concentrations of TNFα were barely detectable in CM from cells of the immunosuppressed subject in contrast to the high levels present in CM from cells of a woman with breast cancer. It is likely, therefore, that TNFα has an important role in regulating aromatase activity.

TNFα, like other cytokines, acts by interacting with cell-surface receptors (10). Using human macrophages, paclitaxel, a compound that stabilises microtubules, was found to rapidly down-regulate TNFα receptors (11). The endogenous oestrogen metabolite, 2-methoxyoestradiol (2-meOE2), was recently shown to have a similar effect to that of paclitaxel on microtubule stability (12, 13).

### SUMMARY OF THE INVENTION

Aspects of the present invention are defined in the appended claims.

In a first aspect the present invention provides use of a material selected from (i) microtubule stabilising agent, which may be one or more of doublecortin, paclitaxel (Taxol), tubercidin, docetaxel (Taxotere), epothilones, (−)-laulimalide, and discodermolide or derivatives thereof; (ii) microtubule disrupter, which may be one or more of human EMAP-like protein-70, paclitaxel (Taxol), colchicine, vinca alkaloids, vinblastine, and nocodzole or derivatives thereof, preferably Paclitaxel; (iii) a compound of the formula A-B wherein A is an oxyhydrocarbyl group and B is a cyclic group, preferably 2-meOE2; and (iv) a compound of the formula C-D wherein C is an sulphamate group and D is a cyclic group, preferably MeOEMATE; for the manufacture of a medicament for the inhibition of tumour necrosis factor α (TNFα) stimulated aromatase activity (23-34).

It is known in the art that paclitaxel (Taxol), functions both as a microtubule stabilizer and a disrupter, depending on the concentration and manner of use. Examples of such use may be found in Lee et al. (1997) and Lloyd and Hardin (1999), see references 23-24.

In the following discussion and throughout the description of the present invention it will be understood by a person skilled in the art that

2-meOE2 is a preferred and is exemplary of a compound of the formula A-B wherein A is an oxyhydrocarbyl group and B is a cyclic group

2-MeOEMATE is a preferred and is exemplary of a compound of the formula C-D wherein C is an sulphamate group and D is a cyclic group. Preferably 2-MeOEMATE is exemplary of a compound of the formula C-D-E wherein C is an sulphamate group, D is a cyclic group, and E is an oxyhydrocarbyl group

Paclitaxel is a preferred and is exemplary of a microtubule disrupter

In the present investigation we have examined the ability of paclitaxel and 2-meOE2 to antagonise TNFα stimulated activity in cultured fibroblasts derived from breast tissues.

The aromatase enzyme, which converts androstenedione to oestrone, regulates the availability of oestrogen to support the growth of hormone-dependent breast tumours. Biological response modifiers, including cytokines, such as interleukin 6 (IL-6) and tumour necrosis factor α (TNFα) or prostaglandin E₂ (PGE₂) can stimulate aromatase activity. These factors may originate from cells of the immune system that infiltrate breast tumours. Paclitaxel, which is used in the treatment of breast cancer, stabilises microtubules and has previously been shown to rapidly down-regulate TNF-receptors on human macrophages. The endogenous oestrogen metabolite, 2-methoxyoestradiol (2-meOE2) also acts to stabilise microtubules. In this study we have examined the ability of paclitaxel, 2-meOE2 and 2-methoxyoestrone-3-O-sulphamate (2-meOEMATE) to antagonise TNFα-stimulated aromatase activity in stromal fibroblasts derived from normal or malignant breast tissues (Additional information regarding 2-MeOemate can be found in Reference 33, the contents of which is herein incorporated by reference in its entirety). Paclitaxel inhibited basal and TNFα-stimulated aromatase activities by 88% and 91% respectively. 2-MeOE2 also reduced basal and TNFα-stimulated aromatase activities by 46% and 56% respectively. 2-MeOEMATE also reduced basal and TNFα-stimulated aromatase activities. Paclitaxel, 2-meOE2 and 2-MeOEMATE also inhibited stimulation of aromatase activity by IL-6 plus its soluble receptor and PGE₂.

The 16α-hydroxylated derivative of 2-meoE2, 2-methoxyoestriol (2-meOE3), which does not bind to microtubules, was less effective at inhibiting TNFα-stimulated aromatase activity. Increased 2-hydroxylation of oestrogens, and subsequent formation of their 2-methoxy derivatives, may be associated with a reduced risk of breast cancer. It is possible that the pathway of oestrogen metabolism may influence the ability of stromal cells to respond to cytokine stimulation.

The cyclic group may be a single ring or it is a polycyclic ring structure. Here, the term “polycyclic” includes fused and non-fused ring structures including combinations thereof.

In one aspect, the cyclic group may contain any one or more of C, H, O, N, P, halogen (including Cl, Br and I), S and P.

At least one of the cyclic groups may be a heterocyclic group (a heterocycle) or a non-heterocyclic group.

At least one of the cyclic groups may be a saturated ring structure or an unsaturated ring structure (such as an aryl group).

Preferably, at least one of the cyclic groups is an aryl ring.

Preferably, Group A and/or Group C and/or Group E is linked or attached to the aryl ring.

If the cyclic group is polycyclic some or all of the ring components of the compound may be fused together or joined via one or more suitable spacer groups.

The polycyclic compound may comprise a number of fused rings. In this aspect the fused rings may comprise any combination of different size rings, such as 3 six-membered rings (6, 6, 6), a six-membered ring, a seven-membered ring and a six-membered ring (6, 7, 6), a six-membered ring and two eight-membered rings (6, 8, 8) etc.

In one aspect, if the cyclic group is polycyclic, Group A and/or Group C and/or Group E are attached to the same ring of the polycyclic compound.

Thus, in accordance with one aspect of the present invention, preferably the compound is a polycyclic compound.

Preferably the polycyclic compound will contain, inclusive of all substituents, no more than 50 about carbon atoms, more usually no more than about 30 to 40 carbon atoms.

The polycyclic compound can comprise at least two ring components, or at least three ring components, or at least four ring components.

Preferably, the polycyclic compound comprises four ring components.

Preferred polycyclic compounds have a steroidal ring component—that is to say a cyclopentanophenanthrene skeleton, or bio-isosteres thereof.

As is well known in the art, a classical steroidal ring structure has the generic formula of:

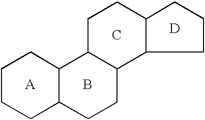

In the above formula, the rings have been labelled in the conventional manner.

An example of a bio-isostere is when any one or more of rings A, B, C and D is a heterocyclic ring and/or when any one or more of rings A, B, C and D has been substituted and/or when any one or more of rings A, B, C and D has been modified; but wherein the bio-isostere in the absence of the sulphamate group has steroidal properties. The ring system ABCD represents a substituted or unsubstituted, saturated or unsaturated steroid nucleus; any one of rings A, B, C or D can have one or more saturated, e.g. double or triple bonds, and can be aromatic, e.g., aryl or phenyl.

In this regard, the structure of a preferred polycyclic compound can be presented as:

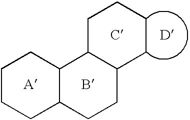

wherein each ring A′, B′, C′ and D′ independently represents a heterocyclic ring or a non-heterocyclic ring, which rings may be independently substituted or unsubstituted, saturated or unsaturated.

By way of example, any one or more of rings A′, B′, C′ and D′ may be independently substituted with suitable groups—such as an alkyl group, an aryl group, a hydroxy group, a halo group, a hydrocarbyl group, an oxyhydrocarbyl group etc.

An example of D′ is a five or six membered non-heterocyclic ring having at least one substituent.

In one preferred embodiment, the ring D′ is substituted with an ethinyl group.

If any one of rings A′, B′, C′ and D′ is a heterocyclic ring, then preferably that heterocyclic ring comprises a combination of C atoms and at least one N atom and/or at least one O atom. Other heterocyclic atoms may be present in the ring.

Examples of suitable, preferred steroidal nuclei rings A′-D′ of the compounds of the present invention include rings A-D of dehydroepiandrosterone and oestrogens including oestrone.

Preferred steroidal nuclei rings A′-D′ of the compounds of the present invention include rings A-D of:

oestrones and substituted oestrones, viz

oestrone

4-OH-oestrone

6α-OH-oestrone

7α-OH-oestrone

16α-OH-oestrone

16β-OH-oestrone

17-deoxyoestrone

oestrone

oestradiols and substituted oestradiols, viz

4-OH-17β-oestradiol

6α-OH-17β-oestradiol

7α-OH-17β-oestradiol

4-OH-17α-oestradiol

6α-OH-17α-oestradiol

7α-OH-17α-oestradiol

16α-OH-17α-oestradiol

16α-OH-17β-oestradiol

16β-OH-17α-oestradiol

16β-OH-17β-oestradiol

17α-oestradiol

17β-oestradiol

17α-ethinyl-17β-oestradiol

17β-ethinyl-17α-oestradiol

17-deoxyoestradiol

oestriols and substituted oestriols, viz

oestriol

4-OH-oestriol

6α-OH-oestriol

7α-OH-oestriol

17-deoxyoestriol

dehydroepiandrosterones and substituted dehydroepiandrosterones, viz

dehydroepiandrosterones

6α-OH-dehydroepiandrosterone

7α-OH-dehydroepiandrosterone

16α-OH-dehydroepiandrosterone

16β-OH-dehydroepiandrosterone

In general terms the ring system A′B′C′D′ may contain a variety of non-interfering substituents. In particular, the ring system A′B′C′D′ may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

In an alternative embodiment, the polycyclic compound may not contain or be based on a steroid nucleus. In this regard, the polycyclic compound may contain or be based on a non-steroidal ring system—such as diethylstilboestrol, stilboestrol, coumarins, flavonoids, combrestatin and other ring systems. Other suitable non-steroidal compounds for use in or as the composition of the present invention may be found in U.S. Pat. No. 5,567,831.

Preferably, Group A or Group C and/or Group E are attached to the same ring of the cyclic compound of the present invention at positions ortho with respect to each other.

Preferably, the polycyclic compound has a steroidal structure and Group A or Group E is attached to the A ring.

Preferably, the Group A or Group E is attached to the 2 position of the A ring of the steroidal structure.

Preferably, the polycyclic compound has a steroidal structure and Group C is attached to the A ring.

Preferably, the Group C is attached to the 3 position of the A ring of the steroidal structure.

Group A is a oxyhydrocarbyl group.

The term “oxyhydrocarbyl group” as used herein means a group comprising at least C, H and O and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the oxyhydrocarbyl group comprises more than one C then those carbons need not necessarily be linked to each other. For example, at least two of the carbons may be linked via a suitable element or group. Thus, the oxyhydrocarbyl group may contain hetero atoms. Suitable hetero atoms will be apparent to those skilled in the art and include, for instance, sulphur and nitrogen.

In one preferred embodiment of the present invention, the oxyhydrocarbyl group is a oxyhydrocarbon group.

Here the term “oxyhydrocarbon” means any one of an alkoxy group, an oxyalkenyl group, an oxyalkynyl group, which groups may be linear, branched or cyclic, or an oxyaryl group. The term oxyhydrocarbon also includes those groups but wherein they have been optionally substituted. If the oxyhydrocarbon is a branched structure having substituent(s) thereon, then the substitution may be on either the hydrocarbon backbone or on the branch; alternatively the substitutions may be on the hydrocarbon backbone and on the branch.

Preferably the oxyhydrocarbyl group is of the formula C₁₋₆O (such as a C₁₋₃O).

If the compound comprises a steroidal nucleus, preferably the A ring has an oxyhydrocarbyl group at the 2 position.

More preferably the group C₁₋₆O is attached to the 2 position of the A ring of a steroidal nucleus.

Preferably, the oxyhydrocarbyl group is an alkoxy.

The alkyl group of the alkoxy substituent is preferably a lower alkyl group containing from 1 to 5 carbon atoms, that is to say methyl, ethyl, propyl etc. Preferably, the alkyl group is methyl.

Thus, in a preferred embodiment, if the compound comprises a steroidal nucleus the A ring has an methoxy substituent at the 2 position.

A preferred compound of the present invention has the formula:

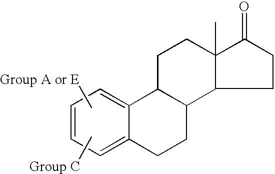

wherein rings A, B, C and D are independently optionally substituted.

Preferably Group A or Group E is in the 2-position.

Preferably Group C is in the 3-position.

Group C is a “sulphamate group”. A “sulphamate group” is a group of the formula

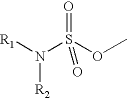

wherein each of R₁ and R₂ is independently selected from H or a hydrocarbyl group.

The term “hydrocarbyl group” as used herein means a group comprising at least C and H and may optionally comprise one or more other suitable substituents. Examples of such substituents may include halo-, alkoxy-, nitro-, a hydrocarbon group, an N-acyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen.

Preferably, R₁ and R₂ are independently selected from H or alkyl, cycloalkyl, alkenyl and aryl, or together represent alkylene, wherein the or each alkyl or cycloalkyl or alkenyl or optionally contain one or more hetero atoms or groups.

When substituted, the N-substituted sulphamate compound may contain one or two N-alkyl, N-alkenyl, N-cycloalkyl, N-acyl, or N-aryl substituents, preferably containing or each containing a maximum of 10 carbon atoms. When R₁ and/or R₂ is alkyl, the preferred values are those where R₁ and R₂ are each independently selected from lower alkyl groups containing from 1 to 5 carbon atoms, that is to say methyl, ethyl, propyl etc. Preferably R₁ and R₂ are both methyl. When R₁ and/or R₂ is aryl, typical values are phenyl and tolyl (-PhCH₃; o-, m- or p-). Where R₁ and R₂ represent cycloalkyl, typical values are cyclopropyl, cyclopentyl, cyclohexyl etc. When joined together R₁ and R₂ typically represent an alkylene group providing a chain of 4 to 6 carbon atoms, optionally interrupted by one or more hetero atoms or groups, e.g. —O— or —NH— to provide a 5-, 6- or 7-membered heterocycle, e.g. morpholino, pyrrolidino or piperidino.

Within the values alkyl, cycloalkyl, alkenyl, acyl and aryl we include substituted groups containing as substituents therein one or more groups which do not interfere with the activity of the compound in question. Exemplary non-interfering substituents include hydroxy, amino, halo, alkoxy, alkyl and aryl. A non-limiting example of a hydrocarbyl group is an acyl group.

In some preferred embodiments, at least one of R₁ and R₂ is H.

Examples of suitable sulphamate compounds for use in the present invention, or examples of suitable compounds that can be converted to suitable sulphamate compounds for use in the present invention, can be found in the art—such as PCT/GB92/01587, PCT/GB97/03352, PCT/GB97/00444, GB 9725749.7, GB 9725750.5, U.S. Pat. No. 5,567,831, U.S. Pat. No. 5,677,292, U.S. Pat. No. 5,567,831, WO-A-96/05216, and WO-A-96/05217.

By way of example, PCT/GB92/01587 teaches novel sulphamate compounds and pharmaceutical compositions containing them for use in the treatment of oestrone dependent tumours, especially breast cancer. These sulphamate compounds are sulphamate esters. Examples of such inhibitors are sulphamate ester derivatives of steroids.

Another compound suitable for use in the present invention has at least the following skeletal structure:

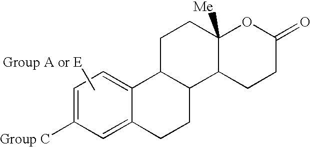

It is preferred that at least one of R₁ and R₂ is H.

Another compound suitable for use in the present invention has at least the following skeletal structure:

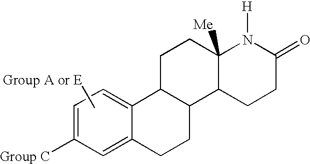

wherein rings A, B, C and D are independently optionally substituted.

A preferred compound of the present invention has the formula:

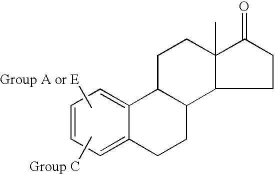

wherein rings A, B, C and D are independently optionally substituted.

Preferably Group E is in the 2-position.

Preferably Group C is in the 3-position.

For the present invention, preferably the sulphamate compound is an oxyhydrocarbyl steroidal sulphamate compound, in particular 2-methoxyoestrone-3-O-sulphamate, or a pharmaceutically active salt thereof, including analogues thereof.

2-methoxyoestrone-3-O-sulphamate is an analogue of oestrone-3-O-sulphamate (otherwise known as “EMATE”), which has the following structure:

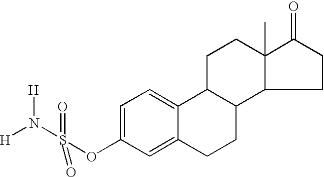

- and can be called 2-methoxy EMATE.

2-methoxy EMATE is the sulphamoylated derivative of a naturally occurring oestrogen metabolite of the present invention, 2-methoxyoestrone. This compound is formed in the liver by the hydroxylation of oestrone by a 2-hydroxylase, with subsequent metabolism to the methoxy derivative by catechol oestrogen methyl transferase.

2-methoxy EMATE has the formula presented as formula below:

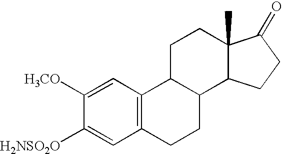

In one embodiment, preferably, the sulphamate compound is an oxyhydrocarbyl derivative of oestrone-3-O-sulphamate.

In one embodiment, preferably, the sulphamate compound is a C₁₋₆ (such as a C₁₋₃) alkoxy derivative of oestrone-3-O-sulphamate.

In one embodiment, preferably, the sulphamate compound is a 2-C₁₋₆ (such as a C₁₋₃) alkoxy derivative of oestrone-3-O-sulphamate.

In one embodiment, preferably, the sulphamate compound is 2-methoxyoestrone-3-O-sulphamate.

The invention will be now be further described by way of example only with reference to the figures which show:

### DESCRIPTION OF THE FIGURES

FIG. 1

Inhibition of basal and TNFα-stimulated aromatase activity by paclitaxel (Pax) in breast tissue fibroblasts. Paclitaxel was added to cells which were cultured for 24 h in 2% stripped foetal calf serum. TNFα was added, in the presence of dexamethasone (100 nmol/l) and cells cultured for a further 48 h in the same medium. Controls and cells with paclitaxel, but not TNFα, were also cultured in the presence of dexamethasone for a 48 h period. Aromatase activity was measured in intact monolayers after washing cells with phosphate buffered saline. (means±SD, n=3; a, p<0.001 versus controls; b, p<0.001 versus TNFα-stimulated aromatase activity).

FIG. 2

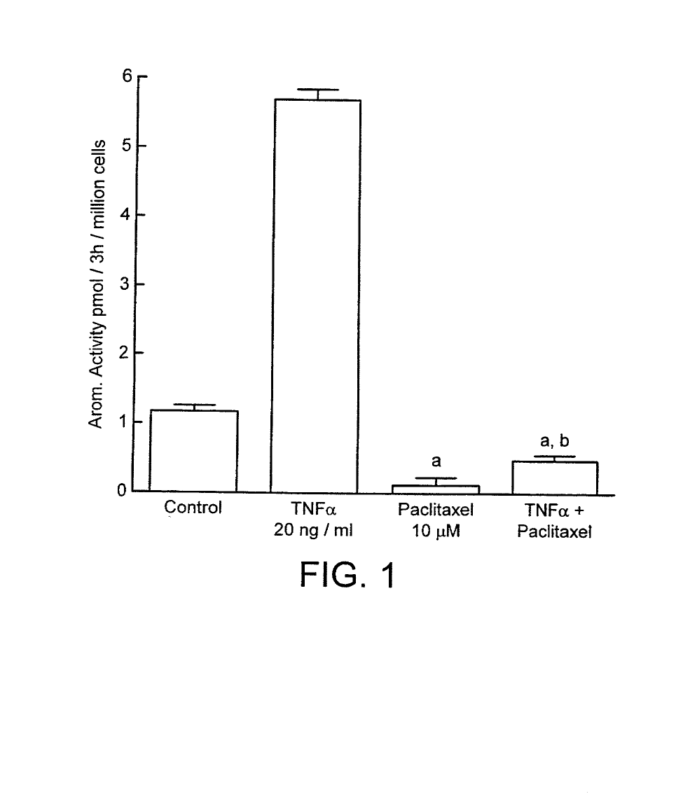

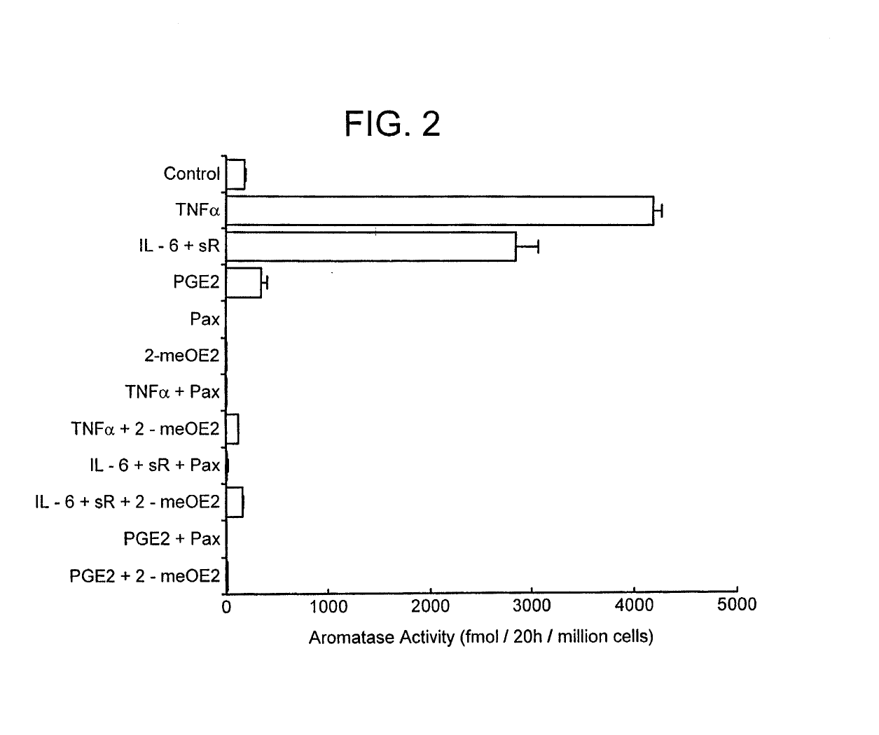

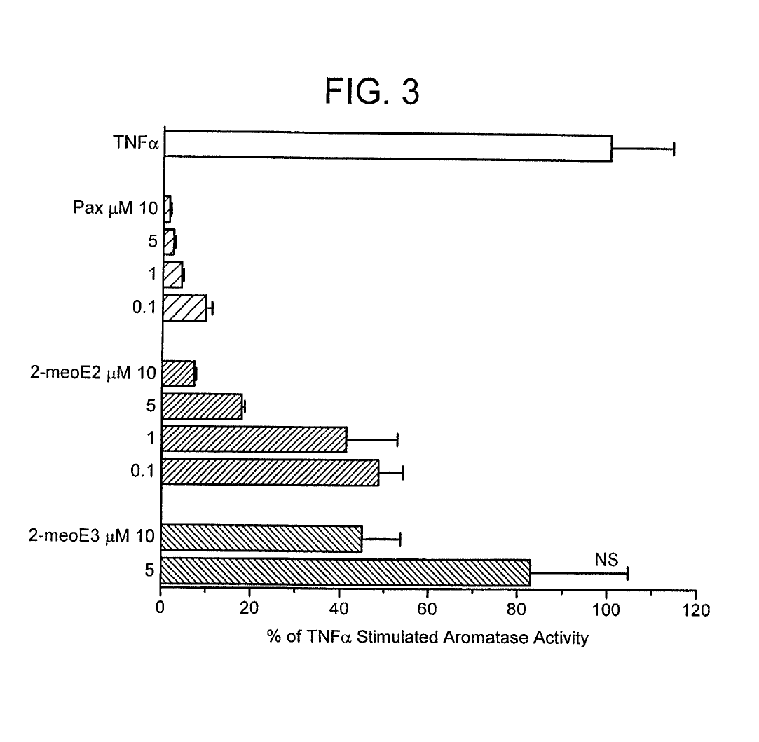

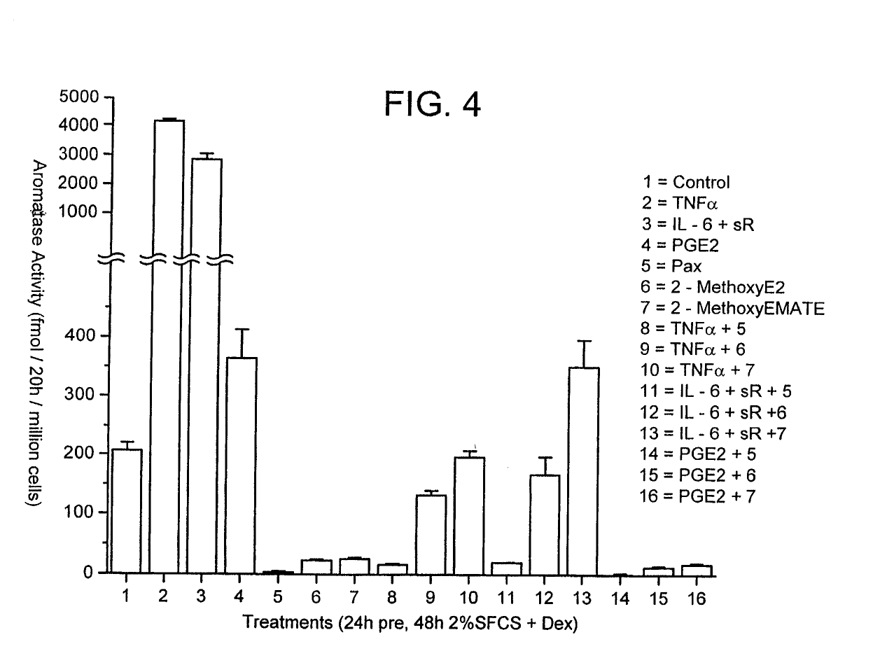

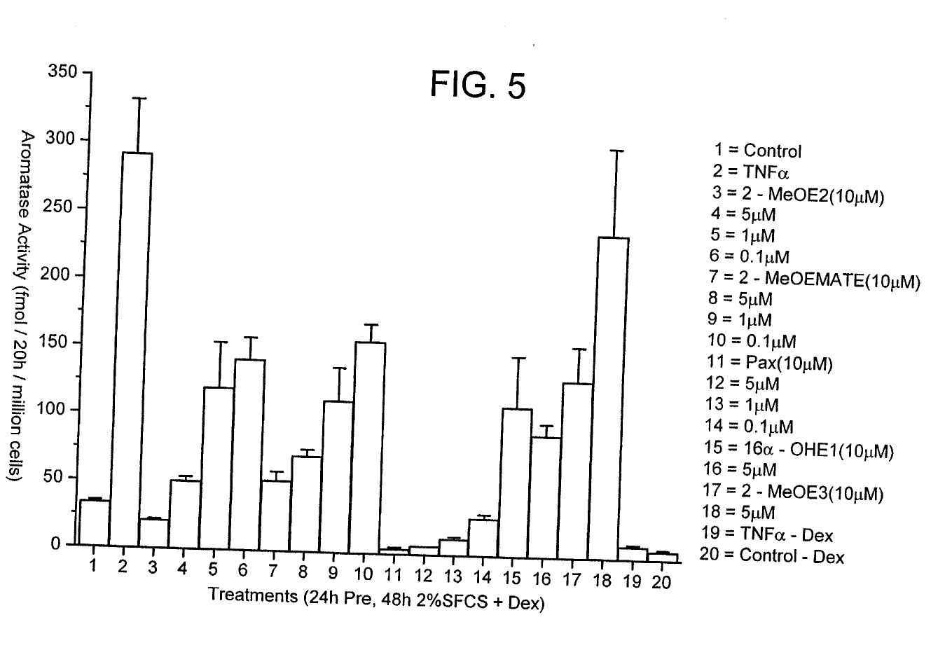

Inhibition of TNFα (20 ng/ml), IL-6 plus IL-6sR (50 ng+100 ng/ml) or PGE₂ (10 μM) stimulated aromatase activity in fibroblasts by paclitaxel (Pax, 10 μM) or 2-methoxyoestradiol (2-meOE2, 10 μM). (means±SD, n=3). The experimental protocol used was as described in the legend to FIG. 1. Aromatase activity in cells treated for 48 h with TNFα, IL-6 plus IL-6sR or PGE₂ was significantly higher (p<0.01-p<0.001) than in control cells. All inhibitions were significant (p<0.001) compared with aromatase activity in TNFα-stimulated cells in the absence of paclitaxel or 2-methoxyoestradiol.

FIG. 3

Dose response for inhibition of TNFα-stimulated aromatase activity (expressed as % of TNFα-stimulated activity) by paclitaxel (Pax), 2-methoxyoestradiol (2-meOE2) or 2-methoxyoestriol (2-meOE3) in fibroblasts. (means±SD, n=3). The experimental protocol used was as described in the legend to FIG. 1. All compounds significantly inhibited TNFα stimulation of aromatase activity (p<0.001) with the exception of 2-methoxyoestriol at 5 μM (NS).

FIG. 4

Ability of compounds to block stimulation of aromatase activity by TNFα, IL-6+IL-6sR or PGE2.

FIG. 5

Dose response for inhibition of TNFα-stimulated aromatase activity.

### EXAMPLES

Materials and Methods

Samples of breast adipose or tumour tissue were obtained from women undergoing reduction mammoplasty or lumpectomy after obtaining their informed consent.

Fibroblasts were cultured as previously described (2). Briefly, they were cultured in Eagles' modified minimal essential medium containing Hepes buffer (20 mmol/l), 10% foetal calf serum (FCS) and supplements. Cells were routinely passaged 2-3 times after which replicate 25 cm² flasks were seeded with fibroblasts and grown to confluency. 2-MeOEMATE was prepared in as described in Appendix I. (For experiments, cells were cultured in phenol red-free medium containing 2% stripped FCS for 24 h in the presence of paclitaxel, 2-meOE2 or 2-MeOEMATE before the addition of TNFα, IL-6 plus IL-6 soluble receptor (IL-6sR) or PGE₂ and cultured for a further 48 h in this medium. TNFα, IL-6 and IL-6sR (R&D Systems Ltd, Abingdon, Oxford, UK) or PGE₂ (Sigma, Poole, Dorset, UK) were used in the presence of dexamethasone (100 nmol/l, Sigma). Paclitaxel, 2-meOE2 and other chemicals were also obtained from Sigma.

At the end of the treatment period aromatase activity was measured in intact monolayers using [1β-³H] androstenedione (15-30 Ci/mmol, NEN-Du Pont, Stevenage, Herts, UK) over a 3-20 h period (2, 3). The number of cells was measured by counting cell nuclei using a Coulter counter. Experiments were carried out in triplicate and results shown are representative of 2-3 investigations.

Statistics

Student's t test was used to assess the significance of differences in mean values of treated and control cells.

Results

The ability of paclitaxel to inhibit TNFα stimulated aromatase activity was initially examined using fibroblasts derived from reduction mammoplasty tissue (FIG. 1). In these cells TNFα, in the presence of dexamethasone, stimulated aromatase activity by 375%. Both paclitaxel and 2-meOE2 inhibited basal aromatase activity by 88% and 46% respectively. In addition, TNFα stimulated aromatase was also significantly reduced by these compounds by 91% and 56% respectively. This ability appears to be specific to agents that stabilise microtubules. Colchicine, which inhibits microtubule polymerisation, or Cytocholasin B, which binds to microfilaments, were without effect (data not shown).

As other factors, such as IL-6 and PGE₂, also act via interaction with cell surface receptors, the ability of paclitaxel, 2-meOE2 and 2-MeOEMATE to antagonise aromatase stimulation by these factors and TNFα was also examined (FIG. 2 and FIG. 4). TNFα, IL-6 plus IL-6sR or PGE₂ all significantly enhanced aromatase activity in tumour-derived fibroblasts. Paclitaxel, 2-meOE2 and 2-MeOEMATE inhibited basal aromatase activity and TNFα stimulated activity. In addition, however, they were also found to antagonise stimulation of aromatase activity by IL-6 plus IL-6sR or PGE₂.

The relative potencies of paclitaxel, 2-meOE2 and 2-MeOEMATE to antagonise TNFα stimulated aromatase activity were compared in a dose-response study (FIG. 3 and FIG. 5). In addition, the ability of the 16α-hydroxy derivative of 2-meOE2, 2-meOE3, which does not appear to bind to microtubules (14), to inhibit TNFα stimulated aromatase activity was also examined.

While paclitaxel, 2-meOE2 and 2-MeOEMATE inhibited TNFα stimulated aromatase, it was evident that paclitaxel is a more potent antagonist. At 0.1 μM paclitaxel inhibited stimulation by 90% whereas 2-meOE2 at this concentration only reduced the stimulation by 51%. 2-MeOE3, while showing some inhibitory effect at the highest concentration tested did not significantly reduce TNFα stimulation of aromatase activity at 5 μM.

Discussion

Results from this investigation have revealed agents that alter microtubule stability, paclitaxel, 2-meOE2 and 2-MeOEMATE, not only inhibit basal aromatase activity but greatly reduce TNFα stimulated activity. Paclitaxel is used in the treatment of breast cancer but, as far as we are aware, this is the first report demonstrating its ability to inhibit basal and cytokine stimulated aromatase activity. This property is restricted microtubule stabilising agents as colchicine or cytocholasin B, which have different effects on the microskeleton, were unable to inhibit TNFα stimulated aromatase activity.

TNFα, IL-6 plus IL6sR and PGE2 are the three main factors identified so far that can regulate aromatase activity in fibroblasts derived from subcutaneous adipose or breast tissues. Microtubules may be required for the synthesis of cytokine receptors or for their translocation to the plasma membrane (15). It is likely, therefore, that the effect that paclitaxel, 2-meOE2 and 2-MeOEMATE have on the ability of TNFα, IL-6 or PGE₂ to stimulate aromatase activity may also result from an effect on the synthesis/translocation of the receptors involved in their signalling. The ability of paclitaxel, 2-meOE2 and 2-MeOEMATE to reduce basal (i.e. unstimulated) aromatase activity may result from blocking the autocrine/paracrine action of cytokines and PGE₂, which are known to be produced by these fibroblasts, on aromatase activity (6, 16).

Paclitaxel is used for the treatment of breast cancer but its toxicity precludes its long-term use. The finding that 2-meOE2, an endogenous oestrogen metabolite, and 2-MeOEMATE may have similar properties to paclitaxel suggests they may have considerable therapeutic potential (17). Oral administration of 2-meOE2 to mice inoculated with B₁₆ melanoma, Meth A sarcoma or MDA-MB-435 breast cancer cells significantly reduced tumour growth (18, 19).

The results from this investigation also suggest a possible mechanism by which the immune system could develop an immunostimulatory role. In the presence of adequate production and/or administration of 2-meOE2 or 2-MeOEMATE, cytokine receptors in breast tissues would be down-regulated and thus cytokine stimulation of aromatase activity inhibited. Reduced production and/or administration of 2-meOE2 or 2-MeOEMATE would enable cytokines to stimulate oestrogen synthesis in breast tissues. Bradlow and his colleagues have obtained convincing evidence that a reduction in the formation of 2-hydroxyoestrogens and an increase in synthesis of 16α-hydroxy metabolites is associated with an increased risk of breast cancer (20, 21). The observation in the present study that the 16α-hydroxy derivative of 2-meOE2 had only a limited ability to suppress TNFα stimulated aromatase activity would appear to support Bradlow's findings.

Stromal fibroblasts cultured from adipose tissue have the ability to differentiate into adipocytes. TNFα, while stimulating aromatase activity in fibroblasts, inhibits their differentiation into adipocytes. High concentrations of oestradiol (10-100 μM) can inhibit TNFα stimulated aromatase activity in adipose stromal cells and it has been postulated that a feed-back loop may exist to prevent excessive oestrogen synthesis in these cells (22). Peroxisome prolierator activated receptor γ (PPARγ) ligands, such as thiozolidinedione, can also stimulate adipocyte differentiation and also inhibit TNFα stimulated aromatase activity. As high concentrations of oestradiol were required to inhibit TNFα stimulated aromatase activity in human adipose stromal cells it is tempting to speculate that oestradiol may act after conversion to 2-meOE2.

Various modifications and variations of the present invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

Synthesis of 2-methoxyoestrone-3-O-sulphamate (2-methoxy EMATE)

2-methoxy EMATE was synthesised by treating a solution of 2 methoxyoestrone in anhydrous dimethylformamide with sodium hydride at 0° C. After evolution of hydrogen had ceased sulphamoyl chloride (2 equiv.) was added and the reaction mixture was allowed to warm to room temperature overnight. The compound was purified by silica gel flash chromatography, was a single pure spot by TLC and exhibited satisfactory spectroscopic and microanalytical data.

In this regard, 2-Methoxy oestrone (75 mg, 0.250 mmol) gave a crude product (103 mg) which was fractionated on silica (50 g) with chloroform/acetone (8:1) and upon evaporation the second fraction gave a pale white residue (83 mg, 81%) which was recrystallized in ethylacetate/hexane (1:2) to give 1 as white crystals (69 mg) .m.p=177-180° C., R_{f}s=0.29 and 0.54 for chloroform/acetone 8:1 and 4:1 respectively and 0.46 and 0.31 for ethylacetate/hexane 2:1 and 1:1 respectively. vmax (KBr) 3400, 3300 (—NH₂), 1610 (C═O), and 1380 (—SO₂N—) cm⁻¹.δ_{H}(CDCl₃) 0.922 (3H, s, C-18-CH₃), 1.24-2.87 (15H, m), 3.88 (3H, s, C-2-OCH₃), 5.0 (2H, br s, exchanged with D₂O,—SO₂NH₂), 6.93 (1H, s, C-1-H) and 7.06 (1H, s, C-4-H). MS: m/z (+ve ion FAB in m-NBA, rel. intensity) 379.1 [100,(M)⁺], 300.0 [25, (M—SO₂NH₂)⁺]. MS: m/z (−ve ion FAB in m-NBA, rel. intensity) 378.0 [100, (M—H)⁻]. Acc. MS: m/z (FAB⁺)=380.1515 C₁₉H₂₆NO₅S requires 380.1532 Found C, 60.0; H, 6.7; N, 3.67; C₁₉H₂₅NO₅S requires C, 60.14; H, 6.64; N, 3.69%.

The present invention will now be further described by the following numbered paragraphs.

1. Use of a material selected from

(i) microtubule stabilising agent;

(ii) microtubule disrupter;

(iii) a compound of the formula A-B wherein A is an oxyhydrocarbyl group and B is a cyclic group; and

(iv) a compound of the formula C-D wherein C is an sulphamate group and D is a cyclic group for the manufacture of a medicament for the inhibition of tumour necrosis factor α (TNFα) stimulated aromatase activity.

2. Use according to paragraph 1 wherein (iii) is a compound of the formula C-D-E wherein C is an sulphamate group, D is a cyclic group, and E is an oxyhydrocarbyl group

3. Use according to paragraph 1 or 2 wherein the cyclic group has a polycyclic ring structure.

4. Use according to paragraph 2 or 3 wherein Group A and/or Group C and/or Group E is linked or attached to the ring.

5. Use according to paragraph 3 or 4 wherein the polycyclic ring structure comprises three six-membered rings.

6. Use according to paragraph 3, 4 or 5 wherein Group A and/or Group C and/or Group E are attached to the same ring of the polycyclic ring structure.

7. Use according to any one of paragraphs 3 to 6 wherein the polycyclic ring structure is a steroidal ring structure

8. Use according to any one of paragraphs 2 to 7 wherein Group A or Group C and/or Group E are attached to the same ring of the cyclic compound of the present invention at positions ortho with respect to each other.

9. Use according to paragraph 7 or 8 wherein Group A or Group E is attached to the 2 position of the A ring of the steroidal structure.

10. Use according to any one of paragraphs 7 to 9 wherein Group C is attached to the 3 position of the A ring of the steroidal structure.

11. Use according to any one of paragraphs 1 to 10 wherein Group A is of the formula C₁₋₆O (such as a C₁₋₃O).

12. Use according to any one of paragraphs 1 to 11 wherein Group A is an alkoxy.

13. Use according to any one of paragraphs 7 to 12 wherein Group A is a methoxy substituent at the 2 position of the steroidal ring structure.

14. Use according to any one of paragraphs 1 to 13 wherein Group C is a group of the formula

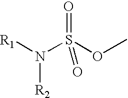

wherein each of R₁ and R₂ is independently selected from H or a hydrocarbyl group.

15. Use according to paragraph 14 wherein R₁ and R₂ are independently selected from H or alkyl, cycloalkyl, alkenyl and aryl, or together represent alkylene, wherein the or each alkyl or cycloalkyl or alkenyl or optionally contain one or more hetero atoms or groups.

16. Use according to paragraph 14 or 15 wherein at least one of R₁ and R₂ is H.

17. Use according to any one of paragraphs 1 to 16 wherein (iv) is a C₁₋₆ (such as a C₁₋₃) alkoxy derivative of oestrone-3-O-sulphamate, preferably a 2-C₁₋₆ alkoxy derivative of oestrone-3-O-sulphamate.

18. Use according to any one of paragraphs 1 to 17 wherein (iv) is 2-methoxyoestrone-3-O-sulphamate.

19. Use according to any one of paragraphs 1 to 18 wherein (ii) is paclitaxel.

20. Use according to any one of paragraphs 1 to 19 wherein (iii) is 2-methoxyoestradiol.

### REFERENCES

1. Reed, M. J., Owen, A. M., Lai, L. C., Coldham, N. G., Ghilchik, M. W., Shaikh, N. A., and James, V. H. T. (1989) Int. J. Cancer 44, 233-237.

2. Reed, M. J., Coldham, N. G., Patel, S., Ghilchik, M. W., and James, V. H. T. (1992) J. Endocrinol. 132, R5-R8.

3. Macdiarmid, F., Wang, D., Duncan, L. J., Purohit, A., Ghilchik, M. W., and Reed, M. J. (1994) Molec. Cell. Endocrinol. 106, 17-21.

4. Zhao, Y., Agarwal, V., Mendelson, C. R., and Simpson, E. R. (1996) Endocrinology 137, 5739-5742.

5. Kelly, P. M., Davison, R. S., Bliss, E., and McGee, J. O. (1988) Br. J. Cancer 57, 174-177.

6. Purohit, A., Ghilchik, M. W., Duncan, L. J., Wang, D. Y., Singh, A., Walker, M. M., and Reed, M. J. (1995) J. Clin. Endocrinol. Metab. 80, 3052-3058.

7. Reed, M. J., and Purohit, A. (1997) Endocrine Rev. 18, 701-715.

8. Stewart, T., Tsai, S.-C. J., Grayson, H., Henderson, R., and Opelz, G. (1995) Lancet 346, 796-798.

9. Singh, A., Purohit, A., Duncan, L. J., Mokbel, K., Ghilchik, M. W., and Reed, M. J. (1997) J. Steroid Biochem. Molec. Biol. 61, 185-192.

10. Tartaglia, L. A., and Goeddel, D. V. (1992) Immunol. Today 13, 151-153.

11. Ding, A. H., Porteu, F., Sanchez, E., and Nathan, C. F. (1990) Science 248, 370-373.

12. Attalla, H., Makela, T. P., Adlercreutz, H., and Andersson, L. C. (1996) Biochem. Biophysc. Res. Comm. 228, 467-473.

13. Attalla, H., Westberg, J. A., Andersson, L. C., Adlercreutz, H., and Makela, T. P. (1998) Biochem. Biophys. Res. Comm. 247, 616-619.

14. Yue, T.-L., Wang, X., Londen, C. S., Gupta, S., Pillarisetti, K., Gu, J.-L., Hart, T. K., Lykso, P. G., and Feuerstein, G. Z. (1997) Molec. Pharmacol. 51, 951-962.

15. Ding, A. H., Porteu, F., Sanchez, E., and Nathan, C. F. (1990) J. Exp. Med. 171, 715-727.

16. Schrey, M. P., and Patel, K. V. (1995) Br. J. Cancer 72, 1412-1419.

17. Zhu, B. T., and Conney, A. H. (1998) Cancer Res. 58, 2269-2277.

18. Fotsis, T., Zhang, Y., Pepper, M. S., Adlercreutz, H., Montesano, R., Nawroth, P. P., and Schweigerer, L. (1994) Nature 368, 273-239.

19. Klauber, N., Parangi, S., Flynn, E., Hamel, E., and D'Amato, R. J. (1997) Cancer Res. 57, 81-86.

20. Bradlow, H. L. Sepkovic, D. W., Telang, N. T., and Osborne, M. P. (1995) Ann. N.Y. Acad. Sci. 728, 180-200.

21. Bradlow, H. L., Telang, N. T., Sepkovic, D. W., and Osborne, M. P. (1996) J. Endocrinol. 150, S259-S265.

22. Simpson, E., Rubin, G., Clyne, C., Robertson, K., O'Donnell, L., Davis, S., and Jones, M. (1999) Endocr. Rel.-Cancer 6, 131-137.

23. Lee, L. F. et al., (1997) Molecular and Cellular Biology 17, 5097-5105.

24. Lloyd, P. G. and Hardin, C. D. (1999) Am. J. Physiol. 277, C1250-C1262.

25. Mooberry, S. L et al., (1995) Cancer Lett. 96, 261-266.

26. Battafarano, D. F. et al., (1995) Cancer 76, 110-115.

27. Bollag, D. M. et al., (1995) Cancer Res. 55, 2325-2333.

28. Nakamura, M. et al., (2000) Am. J. Physiol. Cell Physiol. 279, C480-C487.

29. Paterson, I. et al., (2001) Org. Lett. 3, 3149-3152.

30. Gunasekera, S. P. et al., (2001) J. Nat. Prod. 64, 171-174.

31. Eichenmuller B, et al., (2001) J. Biol. Chem. E-published prior to printing, http://www.jbc.org/cgi/reprint/M106628200v1, PMID: 11694528.

32. Horesh, D. et al., (1999) Hum. Mol. Genet. 8, 1599-1610.

33. MacCarthy-Morrogh, L. et al., (2000) Cancer Research 60, 5441-5450.

1. A composition for the inhibition of tumour necrosis factor α (TNFα) stimulated aromatase activity, wherein the composition comprises at least one of:
(i) microtubule stabilising agent;
(ii) microtubule disrupter;
(iii) a compound of the formula A-B wherein A is an oxyhydrocarbyl group and B is a cyclic group; and
(iv) a compound of the formula C-D wherein C is an sulphamate group and D is a cyclic group.

2. The composition of claim 1, wherein the composition comprises at least one of (i) or at least one of (ii) and at least one of (iii) or at least one of (iv).

3. The composition of claim 1, wherein the composition comprises at least one of (i) or at least one of (ii) and at least one of (iii) and at least one of (iv).

4. The composition of claim 1, wherein (iii) is a compound of the formula C-D-E wherein C is an sulphamate group, D is a cyclic group, and E is an oxyhydrocarbyl group

5. The composition of claim 1, wherein the cyclic group has a polycyclic ring structure.

6. The composition of claim 4, wherein Group A and/or Group C and/or Group E is linked or attached to the ring.

7. The composition of claim 5, wherein the polycyclic ring structure comprises three six-membered rings.

8. The composition of claim 5, wherein Group A and/or Group C and/or Group E are attached to the same ring of the polycyclic ring structure.

9. The composition of claim 5, wherein the polycyclic ring structure is a steroidal ring structure

10. The composition of claim 4, wherein Group A or Group C and/or Group E are attached to the same ring of the cyclic compound of the present invention at positions ortho with respect to each other.

11. The composition of claim 9, wherein Group A or Group E is attached to the 2 position of the A ring of the steroidal structure.

12. The composition of claim 9, wherein Group C is attached to the 3 position of the A ring of the steroidal structure.

13. The composition of claim 1, wherein Group A is of the formula C₁₋₆O (such as a C₁₋₃O).

14. The composition of claim 1, wherein Group A is an alkoxy.

15. The composition of claim 9, wherein Group A is a methoxy substituent at the 2 position of the steroidal ring structure.

16. The composition of claim 1, wherein Group C is a group of the formula 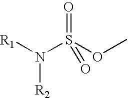
wherein each of R₁ and R₂ is independently selected from H or a hydrocarbyl group.

17. The composition of claim 16, wherein R₁ and R₂ are independently selected from H or alkyl, cycloalkyl, alkenyl and aryl, or together represent alkylene, wherein the or each alkyl or cycloalkyl or alkenyl or optionally contain one or more hetero atoms or groups.

18. The composition of claim 16, wherein at least one of R₁ and R₂ is H.

19. The composition of claim 1, wherein (iv) is a C₁₋₆ (such as a C₁₋₃) alkoxy derivative of oestrone-3-O-sulphamate, preferably a 2-C₁₋₆ alkoxy derivative of oestrone-3-O-sulphamate.

20. The composition of claim 1, wherein (iv) is 2-methoxyoestrone-3-O-sulphamate.

21. The composition of claim 1, wherein (ii) is selected from the group consisting of human EMAP-like protein-70, paclitaxel (Taxol), colchicine, vinca alkaloids, vinblastine, and nocodazole or derivatives thereof.

22. The composition of claim 1, wherein (ii) is paclitaxel.

23. The composition of claim 1, wherein (iii) is 2-methoxyoestradiol.

24. The composition of claim 1, wherein (i) is selected from the group consisting of doublecortin, paclitaxel (Taxol), tubercidin, docetaxel (Taxotere), epothilones, (−)-laulimalide, and discodermolide or derivatives thereof.

25. A method of making a composition for the inhibition of tumour necrosis factor α (TNFα) stimulated aromastase activity, wherein the composition comprises at least one of:
(i) microtubule stabilising agent;
(ii) microtubule disrupter;
(iii) a compound of the formula A-B wherein A is an oxyhydrocarbyl group and B is a cyclic group; and
(iv) a compound of the formula C-D wherein C is an sulphamate group and D is a cyclic group.

26. The composition of claim 25, wherein the composition comprises at least one of (i) or at least one of (ii) and at least one of (iii) or at least one of (iv).

27. The composition of claim 25, wherein the composition comprises at least one of (i) or at least one of (ii) and at least one of (iii) and at least one of (iv).

28. A pharmaceutical composition for the inhibition of tumour necrosis factor α (TNFα) stimulated aromatase activity, wherein the composition comprises at least one of:
(i) microtubule stabilising agent;
(ii) microtubule disrupter;
(iii) a compound of the formula A-B wherein A is an oxyhydrocarbyl group and B is a cyclic group; and
(iv) a compound of the formula C-D wherein C is an sulphamate group and D is a cyclic group.

29. The pharmaceutical composition of claim 28, wherein the composition comprises at least one of (i) or at least one of (ii) and at least one of (iii) or at least one of (iv).

30. The pharmaceutical composition of claim 28, wherein the composition comprises at least one of (i) or at least one of (ii) and at least one of (iii) and at least one of (iv).

31. A method of making a pharmaceutical composition for the inhibition of tumour necrosis factor a (TNFα) stimulated aromatase activity, wherein the composition comprises at least one of:
(i) microtubule stabilising agent;
(ii) microtubule disrupter;
(iii) a compound of the formula A-B wherein A is an oxyhydrocarbyl group and B is a cyclic group; and
(iv) a compound of the formula C-D wherein C is an sulphamate group and D is a cyclic group.

32. The method of claim 31, wherein wherein the composition comprises at least one of (i) or at least one of (ii) and at least one of (iii) or at least one of (iv).

33. The method of claim 31, wherein wherein the composition comprises at least one of (i) or at least one of (ii) and at least one of (iii) and at least one of (iv).

34. A method of treating a patient in need thereof, with the pharmaceutical composition for the inhibition of tumour necrosis factor α (TNFα) stimulated aromatase activity, wherein the composition comprises at least one of:
(i) microtubule stabilising agent;
(ii) microtubule disrupter;
(iii) a compound of the formula A-B wherein A is an oxyhydrocarbyl group and B is a cyclic group; and
(iv) a compound of the formula C-D wherein C is an sulphamate group and D is a cyclic group.

35. The method of claim 34, wherein wherein the composition comprises at least one of (i) or at least one of (ii) and at least one of (iii) or at least one of (iv).

36. The method of claim 34, wherein wherein the composition comprises at least one of (i) or at least one of (ii) and at least one of (iii) and at least one of (iv).
